SEQUENCE LISTING (21 sequences)

SEQ ID NO: 1 - DNA, 104 bp, Artificial Sequence
  Single-stranded DNA fragments Hi-AF designed from the hirudin gene.
gatcctttatggttgtttacactgactgcactgaatccggtcagaacctgtgcctgtgcgaaggctctaacgtttgcggccagggcaacaaatgcatcctgggc

SEQ ID NO: 2 - DNA, 107 bp, Artificial Sequence
  Single-stranded DNA fragments Hi-AR designed from the hirudin gene.
ctctagagcccaggatgcatttgttgccctggccgcaaacgttagagccttcgcacaggcacaggttctgaccggattcagtgcagtcagtgtaaacaaccataaag

SEQ ID NO: 3 - DNA, 111 bp, Artificial Sequence
  Single-stranded DNA fragments Hi-BF designed from the hirudin gene.
tctagaggcgaaaaaaatcaatgcgttactggcgaaggtactccgaaaccgcagtctcacaacgacggcgactttgaagaaatcccggaagaatacctgcaataatagggc

SEQ ID NO: 4 - DNA, 108 bp, Artificial Sequence
  Single-stranded DNA fragments Hi-BR designed from the hirudin gene.
ggccgccctattattgcaggtattcttccgggatttcttcaaagtcgccgtcgttgtgagactgcggtttcggagtaccttcgccagtaacgcattgatttttttcgc

SEQ ID NO: 5 - DNA, 33 bp, Artificial Sequence
  Primer Hi-PCR-AF designed from the hirudin gene.
tcgggatcctttatggttgtttacactgactgc

SEQ ID NO: 6 - DNA, 31 bp, Artificial Sequence
  Primer Hi-PCR-AR designed from the hirudin gene.
gcctctagagcccaggatgcatttgttgccc

SEQ ID NO: 7 - DNA, 38 bp, Artificial Sequence
  Primer Hi-PCR-BF designed from the hirudin gene.
ggctctagaggcgaaaaaaatcaatgcgttactggcga

SEQ ID NO: 8 - DNA, 32 bp, Artificial Sequence
  Primer Hi-PCR-BR designed from the hirudin gene.
catgcggccgccctattattgcaggtattctt

SEQ ID NO: 9 - DNA, 45 bp, Capra hircus
  misc_feature: Signal sequence from a goat beta-casein.
atgaaggtcctcatccttgcctgtctggtggctctggccattgca

SEQ ID NO: 10 - protein, 15 aa, Capra hircus
  MISC_FEATURE: Signal sequence from a goat beta-casein.
MKVLILACLVALAIA

SEQ ID NO: 11 - DNA, 34 bp, Artificial Sequence
  Primer Hir1st5′ for the adding the signal sequence from a goat beta-casein to the 5′-terminal of the hirudin gene.
tggctctggccattgcagttgtttacaccgactg

SEQ ID NO: 12 - DNA, 34 bp, Artificial Sequence
  Primer Hir2nd5′ for the adding the signal sequence from a goat beta-casein to the 5′-terminal of the hirudin gene.
tcatccttgcctgtctggtggctctggccattgc

SEQ ID NO: 13 - DNA, 34 bp, Artificial Sequence
  Primer Hir3rd5′ for adding the signal sequence from a goat beta-casein to the 5′-terminal of the hirudin gene.
tcgctcgagatgaaggtcctcatccttgcctgtc

SEQ ID NO: 14 - DNA, 31 bp, Artificial Sequence
  Primer Hir3′XhoI for adding the signal sequence from a goat beta-casein to the 5′-terminal of the hirudin gene.
tcgctcgagttattgcaggtattcttccggg

SEQ ID NO: 15 - DNA, 243 bp, Hirudo medicinalis
  misc_feature: Sequence of hirudin gene from Genebank accession number of M12693.
atgaaggtcctcatccttgcctgtctggtggctctggccattgcagttgtttacaccgactgcactgaatccggtcagaacctgtgcctgtgcgaaggctctaacgtttgtggccagggcaacaaatgcatcctgggctctgacggcgaaaaaaatcaatgcgttactggcgaaggtactccgaaaccgcagtctcacaacgacggcgactttgaagaaatcccggaagaatacctgcaataa

SEQ ID NO: 16 - DNA, 215 bp, Hirudo medicinalis
  misc_feature: Nucleotide sequence of the full-length DNA fragment of the complete coding sequence of hirudin.
gatcctttatggttgtttacactgactgcactgaatccggtcagaacctgtgcctgtgcgaaggctctaacgtttgcggccagggcaacaaatgcatcctgggctctagaggcgaaaaaaatcaatgcgttactggcgaaggtactccgaaaccgcagtctcacaacgacggcgactttgaagaaatcccggaagaatacctgcaataatagggc

SEQ ID NO: 17 - protein, 67 aa, Hirudo medicinalis
  MISC_FEATURE: Amino acid sequence of the full-length DNA fragment of the complete coding sequence of hirudin.
MVVYTDCTESGQNLCLCEGSNNVCGQGNKCILGSRGEKNQCVTGEGTPKPQSHNDGDFEEIPEEYLQ

SEQ ID NO: 18 - DNA, 24 bp, Artificial Sequence
  Primer palphaLA-forward for transgene screened by PCR.
gcttcctagaaccaacactaccag

SEQ ID NO: 19 - DNA, 21 bp, Artificial Sequence
  Primer palphaLA-Reverse for transgene screened by PCR.
gtcgccgtcgttgtgagactg

SEQ ID NO: 20 - DNA, 27 bp, Artificial Sequence
  Primer pBC1-Forward for transgene screened by PCR.
gattgacaagtaatacgctgtttcctc

SEQ ID NO: 21 - DNA, 26 bp, Artificial Sequence
  Primer pBC1-Reverse for transgene screened by PCR.
catcagaagttaaacagcacagttag

(19) United States
(12) Patent Application Publication
(10) Pub. No.: US 2007/0016969 A1
(43) Pub. Date: Jan. 18, 2007

(54) Expression vector for hirudin and transformed cells and transgenic animals containing said vector

(75) Inventors: Ching-Fu Tu, Chunan Town (TW); Chi-Kai Yang, Chunan Town (TW); Chich-Sheng Lin, Chunan Town (TW); Chon-Ho Yen, Chunan Town (TW); I-Chung Chen, Chunan Town (TW); Shinn-Chih Wu, Chunan Town (TW); Yu-Ling Sun, Chunan Town (TW); Ming-Shing Liu, Chunan Town (TW); Ping-Cheng Yang, Chunan Town (TW)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Animal Technology Institute Taiwan

(21) Appl. No.: 10/727,145

(22) Filed: Dec. 3, 2003

Related U.S. Application Data
(63) Continuation-in-part of application No. 10/053,641, filed on Jan. 18, 2002, now abandoned.

(51) Int. Cl. A01K 67/027; C07H 21/04; C12P 21/06; C07K 14/815

(52) U.S. Cl. 800/14; 800/15; 800/16; 800/17; 800/18; 435/69.1; 435/320.1; 435/325; 536/23.5; 530/350

(57) ABSTRACT

The present invention relates to a nucleic acid construct encoding hirudin and a transgenic mammal whose genome comprises the nucleic acid construct as well as a process for producing hirudin from the transgenic mammal.

The present application is a continuation-in-part application of U.S. Ser. No.10/053,641 filed on 18 Jan. 2002. The specification of which is incorporated herein by reference.

### FIELD OF THE INVENTION

The invention relates to a nucleic acid construct comprising in operable association a casein gene promoter, a signal sequence and a nucleotide fragment encoding hirudin, and a transgenic mammal whose genome comprises the nucleic acid construct, which can exude hirudin-containing milk.

### BACKGROUND OF THE INVENTION

Hirudin is a polypeptide consisting of 65 to 66 amino acids (Dodt, J. et al., 1986, FEBS Lett. 7, 202(2): 373-7) with an anti-thrombotic activity, which is naturally isolated from salivary glands of Hirudo medicinalis. Three hirudin variants, HV1, HV2 and HV3, are known as natural hirudins, which slightly differ from each other with respect of the numbers of amino acids and protein structures. Hirudin can specifically bind to thrombin to inhibit the coagulation activity of thrombin. Therefore, hirudin is useful in treating diseases related to the coagulation activity of thrombin or preventing, alleviating or ameliorating symptoms of the diseases including acute coronary syndromes (Weitz, J. I. and Bates, E. R., 2003, Cardiovasc. Toxicol., 3(1): 13-25).

In earlier years, hirudin was obtained by means of purification and isolation form salivary glands of Hirudo medicinalis. By such means, however, it is difficult to obtain a sufficient amount of hirudin for medical uses. Although hirudin can also be produced in a prokaryotic expression system (such as Escherichia coli), wherein the hirudin is secreted to the periplasmic space, by using a gene recombinant technique, to recover the hirudin, however, it is inevitable to disrupt the bacterial cells and thus the yield of hirudin decreases. In addition, the prokaryotic system lacks a post-translational modification on a polypeptide expressed therein. The biological activity of hirudin produced in such prokaryotic system is not desired. Even in a yeast expression system, the yield and biological activity of hirudin remain low (U.S. Pat. No. 5,866,399; and Courtney, M. et al., 1989, Semin Thromb Hemost., 15(3): 288-292).

Given the above, there is still a need to develop an expression system in which hirudin is produced in a large amount and the produced hirudin is easily recoverable.

### SUMMARY OF THE INVENTION

In one aspect, the invention provides a nucleic acid construct comprising in operable association a casein gene promoter, a signal sequence and a polynucleotide fragment encoding hirudin.

In another aspect, the invention provides a transgenic non-human mammal whose genome comprises the nucleic acid construct of the invention.

In still another aspect, the invention provides a process for producing hirudin comprising the steps of providing the transgenic non-human mammal of the invention, collecting milk from the mammal and recovering hirudin from the milk.

In still another aspect, the invention provides a process for producing hirudin comprising the steps of providing the transgenic non-human mammal of the invention, generating female offspring whose genome comprises the nucleic acid construct of the invention from the transgenic non-human mammal, collecting milk from the female offspring and recovering hirudin from the milk.

In a further aspect, the invention provides an expression vector comprising a replication origin and the nucleic acid construct of the invention.

In a still further aspect, the invention provides a transformed mammal gland cell comprising the expression vector of the invention.

In a still further aspect, the invention provides a process for producing hirudin comprising the steps of culturing the transformed mammal gland cell under the conditions suitable for expressing hirudin and recovering hirudin therefrom.

In still another aspect, the invention provides a process for producing hirudin comprising the steps of isolating mammary gland cells from the transgenic non-human mammal, culturing the isolated mammary gland cells under the conditions suitable for expressing hirudin, and recovering hirudin therefrom.

### BRIEF DESCRIPTION OF DRAWINGS

FIG. 1 shows a synthesis procedure of the polynucleotide fragment encoding hirudin of the invention.

FIG. 2 shows the construction of pE-αLA-Hi of the invention.

FIG. 3 is a plot showing plasma coagulations of a nature hirudin and homogenous extracts of the transformed mammary gland cells and tissue of the invention.

FIG. 4 is a plot showing plasma coagulations of a nature hirudin and culture medium of the transformed mammary gland cells and tissue of the invention.

FIG. 5 shows a PCR analysis of the αLA-hirudin transgenic mouse (A) and pig (B) of the invention. The symbols “+”, “−[, “Tg” and “W” present a positive control, a negative control, the transgenic animals and water, respectively.

FIG. 6 shows the plasmid map of the pBC1-GB-Hir expression vector of the invention.

FIG. 7 shows a PCR analysis of the transgenic (Tg) mice according to the invention. Lane M represents a 1 kb marker; lanes 1 to 11 represent different mouse lines 2-1 (Tg), 2-2, 2-3, 2-4, 4-1, 4-2, 4-3, 4-4 (Tg), 6-1 (Tg), 6-2 and 6-3 (Tg), respectively; lane “+” represents a positive control; lane “−[ represents a negative control and lane “H₂O” represents water as template.

FIG. 8 shows a Southern blotting analysis of the transgenic mice according to the invention. Lane M represents a lambda DNA/HindIII marker; and lanes 1 to 7 represent transgenic mice lines 2-1, 4-4, 6-1, 6-3, NC (that is a normal control i.e., non-transgenic mice genome), one copy (i.e., normal control in combination with one copy of transgenic mice genome) and 10 copies (i.e., normal control in combination with ten copy of transgenic mice genome), respectively.

FIG. 9 (A) is a plot showing a standard curve of the anti-coagulation activity of hirudin.

FIG. 9 (B) is a plot showing the anti-coagulation activity of hirudin in milk of the transgenic mice according to the invention. The curve “_▪_” represents the anti-coagulation activity of normal mouse milk as a negative control. The curve “_□_” represents the anti-coagulation activity of milk of the transgenic mouse milk according to the invention. The curve “_Δ_” represents the anti-coagulation activity of normal mouse milk containing different amounts of nature hirudin (the first point: 50 ng, the second point: 100 ng and the third point: 200 ng) as a positive control.

### DETAILED DESCRIPTION OF THE INVENTION

I. Definitions

The term “construct” used herein refers to a nucleic acid molecule comprising one or more elements e.g., a polynucleotide fragment encoding a protein of interest and a promoter driving the expression of the protein.

The terms “in operable association,” “in operable order” and “operatically linked” used herein refer to the linkage of polynucleotide elements in such a manner that a nucleic acid molecule allowing the transcription of a given gene and/or the synthesis of a desired protein molecule is produced. The terms also refer to the linkage of amino acid sequences in such a manner so that a functional protein is produced.

The term “gene” used herein refers to a polynucleotide fragment that coding sequences essential for the production of a polypeptide or precursor. The polypeptide can be encoded by a full-length coding sequence or by any portion of the coding sequence as long as the desired biological activity is retained.

The term “hirudin” used herein refers to any forms of hirudin or analogs thereof, naturally isolated or artificially synthesized, as long as the desired biological activity is retained.

The term “expression vector” used herein refers to a nucleic aid molecule capable of carrying and transferring a nucleic acid fragment of interest into a host cell for expressing the same. In particular, an expression vector, used in recombinant DNA technology, is a plasmid, cosmid or virus.

The term “host cell” used herein refers to a cell of a host, which can be infected with a vector, such as a plasmid.

The “non-human mammal” used herein refers to any non-human mammal whose genome contains the nucleic acid construct of the invention. Such non-human mammals include, but are not limited to, rodents, non-human primates, sheep, bovines, ruminants, lagomorphs, pigs, goats, equines, canines, felines and aves.

The term “transgene” used herein refers to a foreign gene that is placed into an organism by introducing the foreign gene into an embryonic stem (ES) cells, newly fertilized eggs or early embryos. According to the invention, the transgene refers to a gene or polynucleotide fragment encoding hirudin or analogs thereof.

The terms “promoter element” or “promoter” used herein refer to a DNA sequence that is located at the 5′ end (i.e., upstream) of a gene in a DNA polymer and provides a site for initiation of transcription of the gene into mRNA.

II. Objects of the Invention

A. Nucleic Acid Construct

In one aspect, the invention provides a nucleic acid construct comprising in operable association a casein gene promoter, a signal sequence and a polynucleotide fragment encoding hirudin.

According to the invention, the casein gene promoter of the nucleic acid construct is isolated from a casein gene of mammals, which include but are not limited to human, pig, cattle, horse, goat, camel, sheep or rodent. In one embodiment, the casein gene promoter is isolated from a goat β-casein gene. Many commercially available vectors e.g., pBC1 vector provided by Invitrogen Corporation, can provide a suitable casein gene promoter for constructing the nucleic acid construct of the invention.

The term “signal sequence” used herein refers to an amino acid sequence or its corresponding nucleotide sequence that determines the location of an expressed polypeptide operatically linked to the signal sequence. Indeed, a signal sequence plays an important role on secretion of recombinant protein to milk (Persuy, M. A. et al., 1995, Gene 165(2): 291-6). According to the invention, the signal sequence enhances the secretion of hirudin from mammary gland cells to milk exuded therefrom. The signal sequence of the invention can be derived from a casein gene of mammals, which include but are not limited to human, pig, cattle, horse, goat, camel, sheep or rodent. In one embodiment, the signal sequence of the invention is derived from a goat β-casein gene, preferably a 45 nucleotide sequence of SEQ ID NO: 9.

Nature hirudin and its corresponding gene have been characterized and described in the prior art e.g., those disclosed in the GeneBank under the accession number M12693 (SEQ ID NO: 15). The amino acid sequences of hirudin according to the invention may contain one or more deletions, additions or substitutions of amino acid residues, which result in silent changes and thus do not substantially affect the enzyme activity of the hirudin. Persons skilled in the art can readily obtain the nucleotide sequences of hirudin from the prior art in order to produce a polynucleotide fragment encoding hirudin for preparing the nucleic acid construct of the invention. The detailed procedures for preparing the nucleic acid construct of the invention are described in the following examples.

In one embodiment, the nucleic acid construct further comprises one or more β-globin insulator elements. A β-globin insulator element has been reported to protect a transgene from chromosomal position effects (Chung, J. H. et al., 1997, Proc Natl Acad Sci USA., 94(2): 575-80; and Chung, J. H. et al., 1993, Cell, 74(3): 505-14). It is suggested that the β-globin insulator elements enhance the stability of the nucleic acid construct of the invention when it is inserted into the genome of mammals.

In another embodiment of the invention, the casein gene promoter of the nucleic acid construct is replaced with another promoter isolated from a gene selected from the group consisting of whey acid protein gene, lactoalbumin gene and lactoglobulin gene, more preferably a α-lactoalbumin (α-LA) promoter, of mammals, which include but are not limited to human, pig, cattle, horse, goat, camel, sheep or rodent.

In another aspect, the invention provides a nucleic acid construct comprising a α-LA promoter and a polynucleotide fragment encoding hirudin.

The nucleic acid construct of the invention can be introduced into the genome of a non-human mammal to generate a transgenic non-human mammal capable of secreting hirudin-containing milk.

B. Transgenic Animal

In another aspect, the invention provides a non-human transgenic animal whose genome comprises the nucleic acid construct of the invention.

Recent advances in molecular genetics have provided powerful tools and methods for introducing a gene of interest into the genome of a non-human mammal to generate a transgenic animal, which can be used to study human diseases or produce desired substances. In general, an embryo at various developmental stages may be selected as a target to which a gene of interest is to be introduced. Various methods for introducing a gene of interest into an embryonic cell have been provided depending on the developmental stage of the embryo. For a micro-injection method, it is advantageous to use pronuclear embryos as a target to which a gene of interest is to be introduced. By such means, the injected gene would be incorporated into the genome of the embryo before a fist division of the embryo begins. As a result, all cells of the animal derived from the embryo carry the incorporated gene.

According to the invention, the transgenic non-human mammal is generated by a micro-injection method. In one embodiment of the invention, a female embryo-donor animal is treated by an effective amount of pregnant mare serum gonadotropin (PMSG) and chorionic gonadotropin and subsequently mated with a stud male. Fertilized zygotes are flushed from the oviducts and the pronuclear embryos are injected with the nucleic acid construct of the invention. Survived embryos are transferred into foster dams and the transgenic animals of the invention are born. The detail procedures are described in the following examples. For instance, the genome of the transgenic animals can be confirmed by a PCR or Southern blotting analysis. In one embodiment, the transgenic animals of the invention are non-human transgenic mammals, which include but are not limited to a pig, cattle, horse, goat, camel, sheep or rodent.

The transgene of the transgenic animals of the invention is stably integrated in their germ cells. About 50% of offspring obtain the transgene from their parents. In one embodiment, the germline transmission rate of the transgenic animals of the invention (the number of first generation offspring (F1) whose genomes have the transgene/the total number of first generation offspring) is near 50%.

The transgenic animals or their offspring of the invention can exude hirudin-containing milk from their mammary glands. The hirudin containing milk exuded from the transgenic animals or their offspring exhibit a high level of anti-coagulation activity of hirudin, e.g., ranging from 0.1 u to 40 u per microlitter of the milk. In addition, the anti-coagulation activity of hirudin in the milk remains high during the whole lactation periods.

III. Process for Producing Hirudin

Accordingly, in another aspect, the invention provides a process for producing hirudin, which comprises the steps of providing a female transgenic non-human mammal of the invention, collecting milk exuded from the female transgenic non-human mammal and recovering hirudin from the milk. The invention also provides a process for producing hirudin, which comprises the steps of providing a male transgenic non-human mammal of the invention, generating female offspring whose genome comprises the nucleic acid construct of the invention from the male transgenic non-human mammal, collecting milk from the female offspring and recovering hirudin from the milk.

According to the invention, milk exuded from a female transgenic animal of the invention and female offspring generated from a male transgenic animal of the invention exhibit a high level of biological activity of hirudin, preferably an anti-coagulation activity ranging from 0.1 to 40 u per microlitter of the milk. Hirudin having a high level of biological activity according to the invention can be easily recovered from the milk in light of conventional technology.

In another aspect, the invention provides a mammary gland cell or tissue isolated from the transgenic animal of the invention. The invention also provide a process for producing hirudin comprising the step of isolating mammary gland tissue from the transgenic animal of the invention, culturing the isolated mammary gland tissue under the conditions suitable for expressing of hirudin and recovering hirudin therefrom.

IV. Expression Vector and Transformed Cells

In another aspect, the invention provides an expression vector comprising the nucleic acid construct as described above and a replication origin. According to the invention, the replication origin allows the expression vector to replicate in a mammal cell, preferably a mammal gland cell.

The term “expression vector” used herein refers to a nucleic aid molecule capable of carrying and transferring a nucleic acid fragment encoding a polypeptide of interest into a host cell in order to express the same. Generally, an expression vector, used in recombinant DNA technology, refers to a plasmid or virus.

According to the invention, the expression vector is used for expressing hirudin in a mammal cell, preferably a mammal gland cell. The elements of the expression vector e.g., a casein gene promoter or a α-LA promoter, a signal sequence, a polynucleotide fragment encoding hirudin and one or more β-globin insulators, are descried as above. Preferably, the expression vector of the invention further comprises a selection marker. More preferably, the expression vector of the invention further comprises a tag sequence such that a fused polypeptide is produced and beneficial to the subsequent purification procedures.

The genetic recombination methods involved in the invention including primer design, DNA amplification by PCR, vector construction, cell transformation, and protein expression can be accomplished by persons skilled in the art and which can be seen, for instance, in Molecular Cloning: A Laboratory Manual, Cold Spring Harbor, 1989.

The expression vector of the invention can be introduced into a mammal cell to express hirudin. Accordingly, the invention also provides a mammal cell, preferably a mammal gland cell, transformed with the expression vector of the invention for expressing hirudin therein. A number of transformation methods, including a calcium chloride treatment, calcium-PEG procedure, electroporation, DEAE-dextrin- and liposome-mediated transfection, and microinjection, are well described in the prior art.

Accordingly, the invention further provides a process for producing hirudin, which comprises culturing the transformed mammal cells of the invention under a condition suitable for expressing hirudin and recovering hirudin therefrom.

The present invention will become apparent with reference to the following examples. The examples described below are given by way of illustration only and are not regarded as any limitation of the present invention.

### Examples

### Example 1

### Synthesis of Full-Length DNA Fragment Encoding Hirudin

Based on the sequence of hirudin gene disclosed in the Genebank under the accession number of M12693 (SEQ ID NO: 15), four single-stranded DNA fragments, Hi-AF (SEQ ID NO: 1), Hi-AR (SEQ ID NO: 2), Hi-BF (SEQ ID NO: 3) and Hi-BR (SEQ ID NO: 4), were designed, wherein Hi-AF and Hi-AR are complementary to each other and Hi-BF and Hi-BR are complementary to each other (Table 1). In addition, four primers, Hi-PCR-AF (SEQ ID NO: 5), Hi-PCR-AR (SEQ ID NO: 6), Hi-PCR-BF (SEQ ID NO: 7) and Hi-PCR-BR (SEQ ID NO: 8), were designed according to the 5′-terminal sequences of the above four DNA fragments (Table 1).

TABLE 1

Sequences of hirudin gene M26726,
DNA fragments and primers

| Hirudin | | |
|---|---|---|
| Gene, DNA | | SEQ |
| Fragments | | ID |
| and Primers | Sequences (5′-3′) | NO |
| M12693 | ATGAAGGTCCTCATCCTTGCCTGTCTGGTGGCTC | 15 |
| | TGGCCATTGCAGTTGTTTACACCGACTGCACTGA | |
| | ATCCGGTCAGAACCTGTGCCTGTGCGAAGGCTCT | |
| | AACGTTTGTGGCCAGGGCAACAAATGCATCCTGG | |
| | GCTCTGACGGCGAAAAAAATCAATGCGTTACTGG | |
| | CGAAGGTACTCCGAAACCGCAGTCTCACAACGAC | |
| | GGCGACTTTGAAGAAATCCCGGAAGAATACCTGC | |
| | AATAA | |
| Hi-AF | gatcctttatggttgtttacactgactgcactga | 1 |
| | atccggtcagaacctgtgcctgtgcgaaggctct | |
| | aacgtttgcggccagggcaacaaatgcatcctgg | |
| | gc | |
| Hi-AR | ctctagagcccaggatgcatttgttgccctggcc | 2 |
| | gcaaacgttagagccttcgcacaggcacaggttc | |
| | tgaccggattcagtgcagtcagtgtaaacaacca | |
| | taaag | |
| Hi-BF | tctagaggcgaaaaaaatcaatgcgttactggcg | 3 |
| | aaggtactccgaaaccgcagtctcacaacgacgg | |
| | cgactttgaagaaatcccggaagaatacctgcaa | |
| | taatagggc | |
| Hi-BR | ggccgccctattattgcaggtattcttccgggat | 4 |
| | ttcttcaaagtcgccgtcgttgtgagactgcggt | |
| | ttcggagtaccttcgccagtaacgcattgatttt | |
| | tttcgc | |
| Hi-PCR-AF | Tcgggatcctttatggttgtttacactgactgc | 5 |
| Hi-PCR-AR | gcctctagagcccaggatgcatttgttgccc | 6 |
| Hi-PCR-BF | ggctctagaggcgaaaaaaatcaatgcgttactg | 7 |
| | gcga | |
| Hi-PCR-BR | catgcggccgccctattattgcaggtattctt | 8 |
| Hi | GATCCTTT ATG GTT GTT TAC ACT GAC | 16 |
| nucleotide | TGC ACT GAA TCC GGT CAG AAC CTG | |
| sequence | TGC CTG TGC GAA GGC TCT AAC GTT | |
| | TGC GGC CAG GGC AAC AAA TGC ATC | |
| | CTG GGC TCT AGA GGC GAA AAA AAT | |
| | CAA TGC GTT ACT GGC GAA GGT ACT | |
| | CCC AAA CCG CAG TCT CAC AAC GAC | |
| | GGC GAC TTT GAA GAA ATC CCG GAA | |
| | GAA TAC CTG CAA TAA TAGGGC | |
| Hi | Met Val Val Tyr Thr Asp Cys Thr | 17 |
| amino acid | Glu Ser Gly Gln Asn Leu Cys Leu | |
| sequence | Cys Glu Gly Ser Asn Val Cys Gly | |
| | Gln Gly Asn Lys Cys Ile Leu Gly | |
| | Ser Arg Gly Glu Lys Asn Gln Cys | |
| | Val Thr Gly Glu Gly Thr Pro Lys | |
| | Pro Gln Ser His Asn Asp Gly Asp | |
| | Phe Glu Glu Ile Pro Glu Glu Tyr | |
| | Leu Gln | |

PCR was carried out respectively, by using the DNA fragments Hi-AF and Hi-AR as templates and Hi-PCR-AF and Hi-PCR-AR as primers to amplify the first DNA fragment (Hi-A), and the DNA fragments Hi-BF and Hi-BR as templates and Hi-PCR-BF and Hi-PCR-BR as primers to amplify the second DNA fragment (Hi-B). The above templates (1 ng each), primers (0.2 μM each), 10-fold buffer (10 μl, comprising 15 mM MgCl₂, 500 mM KCl, 1% Triton X-100, 0.1% gelatin and 100 mM Tris-HCl, pH 7.9), dATP, dCTP, dTTP and dGTP (200 μM each), and polymerase (0.5 U; Promega Co., USA) were mixed to a final volume of 100 μl. The reaction mixture was heated at 94° C. for 5 minutes before entering the PCR cycles. The reaction conditions are 94° C. for 45 seconds, 60° C. for 45 seconds and 72° C. for 45 seconds. After a total of 40 cycles, the mixtures were subjected to 72° C. for 3 minutes to complete the DNA extension.

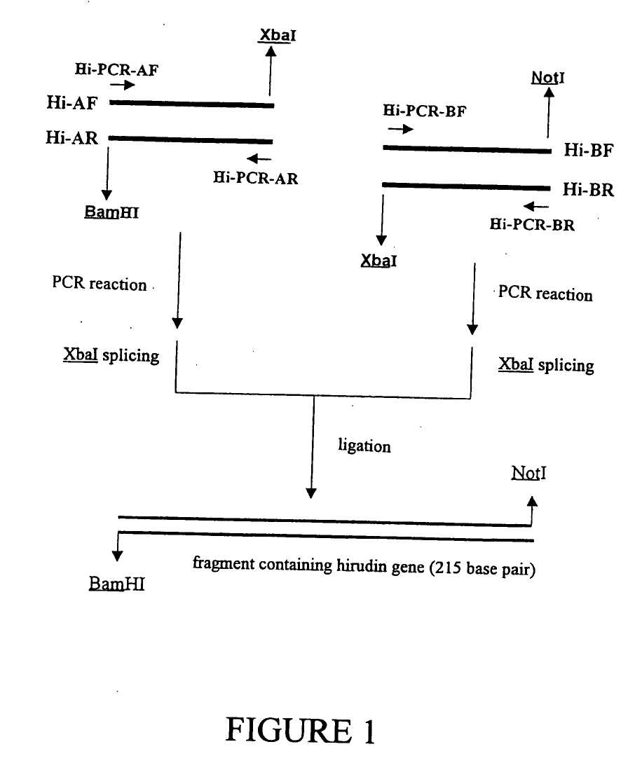

As shown in FIG. 1, a cutting site for the restriction enzyme XbaI was designed at the 3′-terminal of Hi-A and 5′-terminal of Hi-B. The amplified products Hi-A and Hi-B were purified and recovered with a purification kit (PCR Clean Up-M; Viogene) and then treated by the restriction enzyme, XbaI. The enzyme-treated DNA fragments Hi-A and Hi-B were analyzed by electrophoresis and recovered from 2% agarose gel by Gel Extraction Kit (Viogene). The two fragments were ligated to obtain a full-length DNA fragment, designated as “Hi”, containing a complete coding sequence of hirudin. The nucleotide sequence (SEQ ID NO: 16) and amino acid sequence (SEQ ID NO: 17) of the full-length DNA fragment (Hi), respectively, were analyzed and shown in Table 1, wherein amino acid 34 (arginine) is different from the corresponding amino acid (aspartic acid) of HV1 hirudin. The above-mentioned enzyme cutting reaction and ligation were conducted by known standard methods (Current Protocols in Molecular Biology, Eds Frederick M. A., et al., 2001. John Wiley & Sons, Inc. ).

### Example 2

### Construction of Expression Vector

2.1 pBC1-GB-Hir

A signal sequence isolated from a goat β-casein was added to the 5′-terminal of the hirudin gene (SEQ ID NO: 15) by conducting three sequential PCR using three pairs of primers Hir1st5′/Hir3′XhoI, Hir2nd5′/Hir3′XhoI and Hir3rd5′/Hir3′XhoI. The nucleotide sequences of the signal sequence and primers are shown in Table 2.

TABLE 2
Sequences of signal sequence and primers

| Signal | | |
|---|---|---|
| Sequence | | |
| and Primers | Sequences (5′-3′) | SEQ ID NO |
| Signal | ATGAAGGTCCTCATCCTTGCCTGTCTGGT | 9 |
| Sequence | GGCTCTGGCCATTGCA | |
| | MKVLILACLVALAIA | 10 |
| Hir1st5′ | TGGCTCTGGCCATTGCAGTTGTTTACACC | 11 |
| | GACTG | |
| Hir2nd5′ | TCATCCTTGCCTGTCTGGTGGCTCTGGCC | 12 |
| | ATTGC | |
| Hir3rd5′ | TCGCTCGAGATGAAGGTCCTCATCCTTGC | 13 |
| | CTGTC | |
| Hir3′XhoI | TCGCTCGAGTTATTGCAGGTATTCTTCCG | 14 |
| | GG | |

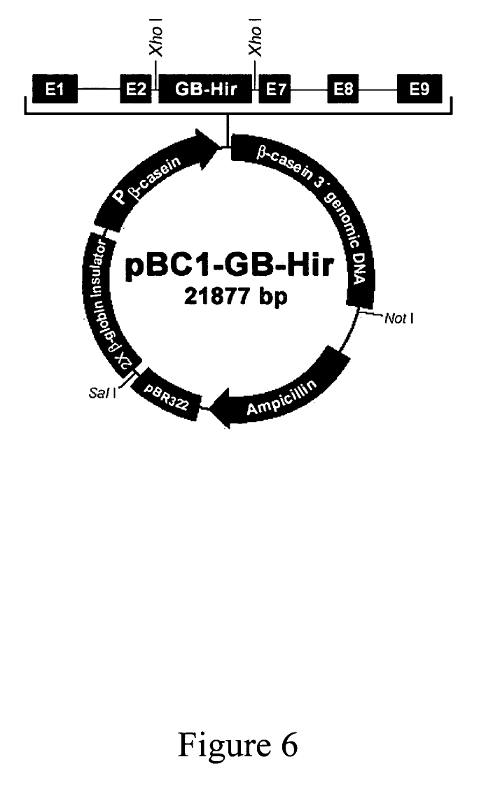

The PCR product was ligated into pCR2. 1 vector to produce pCR2.1-GB-Hir. Then the pCR2.1-GB-Hir was digested with XhoI. A 261 bp GB-Hir fragment thus produced was purified and subcloned into XhoI and alkaline phosphatase treated pBC1 vector to yield an expression vector pBC1-GB-Hir. FIG. 6 shows the plasmid map of pBC1-GB-Hir.

2.2 pE-αLA-Hi

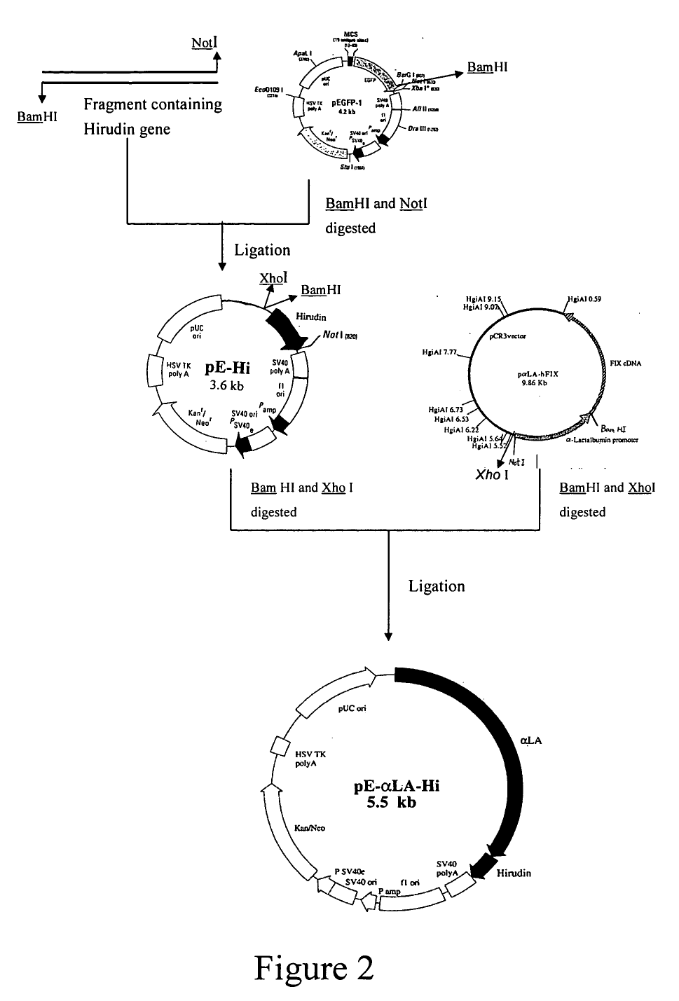

As shown in FIG. 2, two cutting sites for the restriction enzyme BamHI and NotI were designed at 3′-terminal of Hi-A and 5′-terminal of Hi-B, respectively. The full-length DNA fragment (SEQ ID NO: 16) encoding hirudin obtained in Example 1 was treated by the restriction enzymes BamHI and NotI. The enzyme-treated DNA fragment (215 bp) was isolated by electrophoresis and recovered from 2% agarose gel by Gel Extraction Kit. The purified DNA fragment was ligated to a BamHI/NotI treated vector pEGFP-1 (pEGFP-N1 without the EGFP sequence fragment) to obtain an expression vector pE-Hi (3.6 kb). The pE-Hi expression vector was transformed into E. coli NM522 competent cells and the ampicillin resistant transformants were selected. The above transformation employs a known method (Current Protocols in Molecular Biology, Eds Frederick M. A., et al., 2001. John Wiley & Sons, Inc.). The pE-Hi expression vector was amplified by the E. coli transformants and purified for the subsequent procedures.

The purified expression vector pE-Hi was cut by the restriction enzymes BamHI and XhoI to produce a 3.6 kb DNA fragment, which was isolated by electrophoresis and recovered form 1% agarose gel. A 1.9 kb DNA fragment containing a α-lactoalbumin (αLA) promoter that is recovered from BamHI/XhoI treated pαLA-hFIX (S. P. Lin, Construction and Expression of hybrid gene contained the promoter of α-lactoalbumin and the cDNA of human blood clotting factor IX, Master's Thesis of Department of Animal Science of National Taiwan University, 1996) was ligated to the enzyme-treated 3.6 kb DNA fragment to obtain an expression vector, pE-αLA-Hi, which can specifically express hirudin in mammary gland cells.

### Example 3

### Preparation of Transgene

3.1 BC1-GB-Hir

The pBC1-GB-Hir expression vector described in Example 2 was digested with NotI and SalI restriction enzymes to yield a 16 kb DNA construct (BC1-GB-Hir) containing the goat β-casein promoter, the signal sequence and the full-length DNA fragment (SEQ ID NO: 15) encoding hirudin as described above.

The 16 kb DNA construct was separated by electrophoresis and recovered from a low melting point agarose gel. Further purification was conducted by CsCl₂ banding and TE buffer (10 mM Tris-HCl, 0.1 mM EDTA, pH 7.4) dialysis. The purified DNA construct, diluted with TE buffer to a final concentration of 2 to 4 ng/νl, was used as a transgene for mammal pronuclear microinjection.

3.2 αLA-Hi

The pE-αLA-Hi expression vector obtained in Example 2 was digested with restriction enzymes, ClaI and DraIII, to yield a 4.77 kb DNA construct containing the above-described αLA promoter and full-length DNA fragment (SEQ ID NO: 16) encoding hirudin and a SV40 poly A tail sequence. The 4.77 kb DNA construct was separated by electrophoresis and recovered from 1% agarose gel by Gel Extraction Kit and then diluted with TE buffer (10 mM Tris-HCl, 0.25 mM EDTA, pH 7.4) to a final concentration of 1 ng/μl, which was used as a transgene for mammal pronuclear microinjection.

### Example 4

### Generation of Transgene Animal

Mature ICR mice or Landrace pigs were provided as embryo donors and recipients for generating transgenic animals.

4.1 Procedures for Generating Mice

All mice were reared in a clean laboratory rodent house, maintained at 20 to 26° C. and ventilated via a HEPA system with 10-hour dark and 14-hour light period. Fresh water and feed were supplied ad libitium. Each female mouse was superovulated by intraperitoneal injection with PMSG After 48 hours following the PMSG injection, the mice were injected with human chorionic gonadotropin (hCG) and mated with a stud ICR male at the same day. Fertilized zygotes were flushed from the oviducts and the pronuclear embryos were micromanipulated by the Narishige manipulator with a differential interference contrast inverted microscope. The transgene prepared as above was injected into the male pronucleus of mouse embryos and those survived were grouped in 25 to 30 and transferred into the fallopian tubes of foster dams. After laboring, newborn animals were nursed for 4 weeks and then a small piece of tail was cut for extraction of genomic DNA in order to screen the exogene by PCR.

4.2 Procedures for Generating Transgenic Pigs

Pure breed Landrace (L) gilts, at least seven and half-month old, were provided. The animals were fed with 1.0 to 1.2 kg commercial feed twice a day and had access to water ad libitium. Lactation sows were fed with lactation feed. The transgenic piglets were weaned at 28 days after delivery.

All embryo donor and recipient gilts were synchronized by feeding commercial feed mixed with Regumate® (containing 0.4% altrenogest; 20 mg/day; Intervet, Boxmeer, Netherlands) in the morning for 15 days. At 24 hrs following the last feeding of Regumate®, the pigs were superovulated by intramuscularly injecting with PMSG (1500-2000 IU, Intervet, Boxmeer, Netherlands). After 76 to 78 hours following the PMSG injection, the pigs were injected with hCG (1250-1750 IU, Intervet, Boxmeer, Netherlands), and at 24 to 36 hours following the hCG injection, the donors were subjected to artificial insemination with pure breed L boar fresh-diluted semen.

At 54 to 56 hours following the hCG injection, a surgical operation was performed on the donor pigs to flush fertilized zygotes from the fallopian tubes into a dish with 20 ml Dubacos-PBS (purchased from Gibco/BRL, USA) with 0.4% BSA (purchased from Fraction V, Sigma, USA). Before operation, the pigs were fasted overnight and calmed by intramuscularly injecting with 5 ml sterinil (2 mg/100 kg, Janssen Pharmaceutical, Belgium) and 10 ml atropine sulfate (5 mg/100 kg, China Chem. and Pharm., Taiwan), and then initially anaesthetized by injection with sodium pentobarbitone (10 mg/kg, Abbott Australasia Pty Ltd., Australia) at ear veins. The anaesthesia was maintained throughout the operation by 4% halothane (ICI Ltd., USA) inhalation. Embryos were surgically transferred into the fallopian tube of other synchronized foster pigs with the same procedures as for donors. Upon the fallowing, a small piece of the piglet's ear or tail tissue was taken to extract their genomic DNA for analysis.

The fertilized zygotes were centrifuged with 23,500×g for 8 minutes at room temperature by centrifuge (Hettich EBA 12, Germany) to expose the pronuclei. The pig embryos were micromanipulated by Leica mechanical manipulator with differential interference contrast inverted microscope (ZEISS Axiovert 135, Germany). The transgenes were injected into pronuclei of new fertilized zygotes or nuclei of two-cell stage of pig embryos. After 25 to 30 pig embryos were injected, the embryos were transferred into the fallopian tubes of the recipient-synchronized pigs as soon as possible.

4.3 Transgenic Mice by BC1-GB-Hir

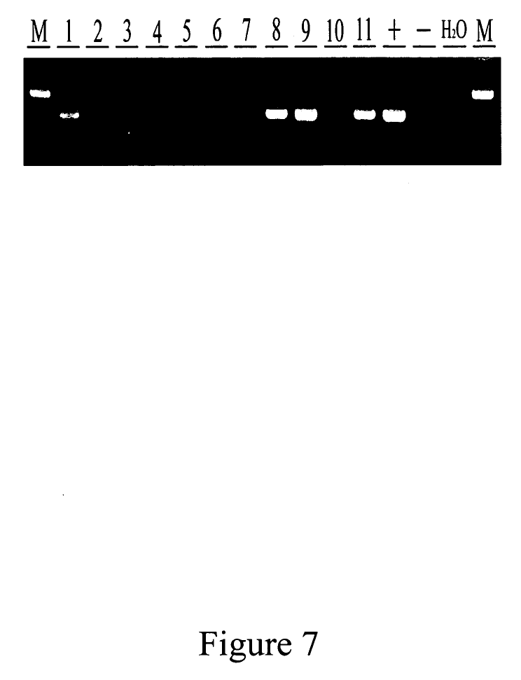

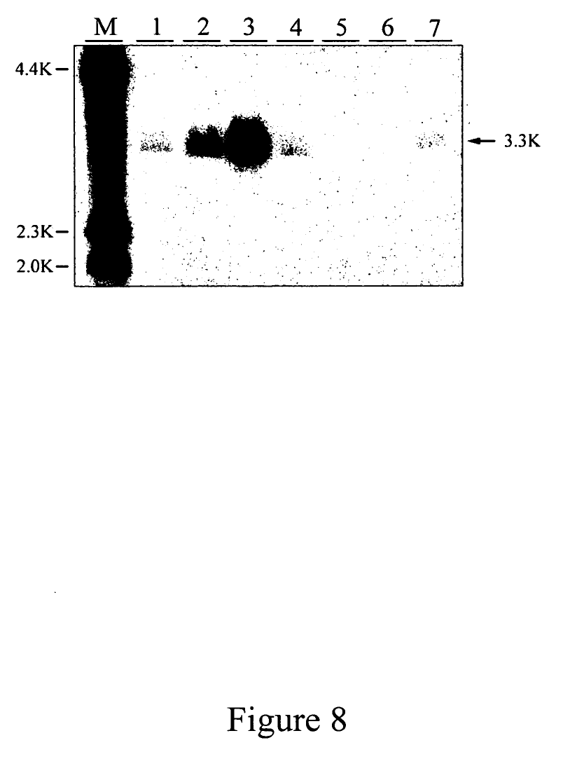

After microinjection of the BC1-GB-Hir transgene, a total of 275 mouse embryos were transferred into the fallopian tube of 10 recipient mice. Seven mice were pregnant and 29 newborn mice were born. Among them, four newborn mice, three male and one female, were confirmed as transgenic mice by a PCR analysis (FIG. 7) and Southern blot hybridization (FIG. 8), which are described below. Although the BC1-GB-Hir construct of the invention is 16 kb in length, the transgene still integrated into mouse embryo genome in a normal percentage. The successful rate of 13.8% (4/29) indicated that the length of transgene causes little interfere to the integration of foreign DNA into embryo genomes.

In addition, all transgenic mice were bred with non-transgenic mice whenever they reached sexual maturity. About 48.4% to 60.0% of the offspring inherited the transgene from their parents. Such a high germline transmission rates indicate a stable integration of the transgene in the germ cells (Table 3).

TABLE 3
Germ-line transmission rate of BC1-GB-Hir transgene

| Founder of | | | No. of F1 | Germ-line |
|---|---|---|---|---|
| transgenic | | No. of F1 | transgenic | transmission |
| mice | Gender | offspring born | offspring | rate (%) |
| 2-1 | male | 28 | 14 | 50.0 (14/28) |
| 4-4 | female | 21 | 11 | 52.4 (11/21) |
| 6-1 | male | 25 | 15 | 60.0 (15/25) |
| 6-3 | male | 31 | 15 | 48.4 (15/31) |

The BC1-GB-Hir construct contains 2×β-globin insulator elements. It is suggested that the β-globin insulator elements enhance the stability of the BC1-GB-Hir construct of the invention when it is inserted into the genome of mammals.

4.4 Transgenic Mice and Pigs by αLA-Hi

As showed in Table 4, 383 mice embryos and 180 pig embryos were injected and transferred into 15 and 8 foster dams, respectively. After pregnancy, 30 newborn mice and 18 piglets were born wherein five mice and one pig were confirmed as transgenic animals.

TABLE 4
Generation of αLA-hirudin transgenic mice and pigs

| | No. of | | No. of | | No. of newborn | |
|---|---|---|---|---|---|---|
| | embryos | | foster | | mice or piglets | |
| | micro- | embryo | In- | Pregnant | | Transgenic |
| Animal | injection | transfer | jection | (%) | Born | (%) |
| Mouse | 563 | 383 | 15 | 10 (66.7) | 30 | 5 (16.7) |
| Pig | 180 | 180 | 8 | 4 (50.0) | 18 | 1 (5.6) |

### Example 5

### Analysis of Genome of Transgene Animal

After delivering newborn mice or piglets, the tail tissues of newborn mice or ear tissues of piglets, respectively, were taken to extract genomic DNAs as PCR template at the weaning or delivery day.

5.1 BC1-GB-Hir

The transgene was screened by PCR with the following pBC1 forward and reverse primers (Table 5).

TABLE 5
Nucleotide sequence of primers

| | | |
|---|---|---|
| Primers | Sequence (5′-3′) | SEQ ID NO |

| | | |
|---|---|---|
| pBC1-Forward | GATTGACAAGTAATACGCTTTTCCTC | 20 |
| pBC1-Reverse | CATCAGAAGTTAAACAGCACAGTTAG | 21 |

Template DNAs (100 ng each) or BC1-GB-Hir (1 ng, positive control) were added into respective PCR reaction mixture and heated at 95° C. for 5 minutes before entering the PCR cycles. The reaction conditions are 95° C. for 30 seconds, 55° C. for 30 seconds and 72° C. for 30 seconds. After a total of 35 cycles, the mixtures were subjected to 72° C. for 2 minutes to complete the DNA extension. Then, the PCR products were analyzed by electrophoresis in a 2% agarose gel and a 385 bp DNA band was observed.

In addition, fifteen microgram genomic DNA were digested with EcoRI restriction enzyme and then analyzed by electrophoresis in a 0.8% agarose gel. After alkali denaturation, the genomic DNA was blotted to a nitrocellulose membrane and hybridized with a P³²-labeled Hirudin DNA probe and revealed by autoradiography.

5.2 αLA-Hi

The transgene was screened by PCR with the following specific primer pair, which was designated according to the up strand of αLA promoter sequence and the down strand of hirudin sequence (Table 6).

TABLE 6
Nucleotide sequence of primers

| | | |
|---|---|---|
| Primers | Sequence (5′-3′) | SEQ ID NO |

| | | |
|---|---|---|
| pαLA-Forward | GCTTCCTAGAACCAACACTACCAG | 18 |
| pαLA-Reverse | GTCGCCGTCGTTGTGAGACTG | 19 |

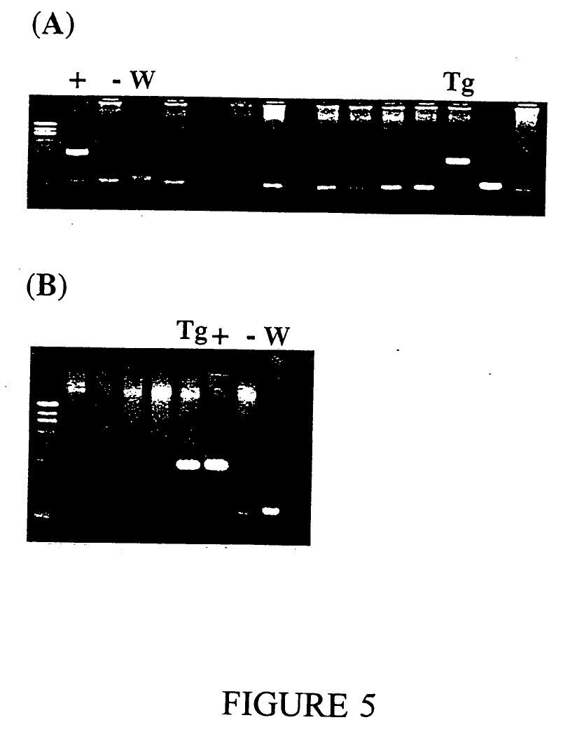

Template DNAs (100 ng each) or pE-αLA-Hi (1 ng, positive control) was add to respective PCR reaction mixture which contains 10-fold PCR buffer (10 μl, comprising 15 mM MgCl₂, 500 mM KCl, 1% Triton X-100, 0.1% gelatin and 100 mM Tris-HCl, pH 7.9), dATP, dCTP, dTTP and dGTP (200 μM each), the above-described primer pair (0.2 μM each) and 0.5 U Tag polymerase. The PCR mixtures were heated at 94° C. for 3 minutes before entering the PCR cycles. The reaction conditions are 94° C. for 60 seconds, 55° C. for 60 seconds and 72° C. for 60 seconds. After a total of 35 cycles, the mixtures were subjected to 72° C. for 3 minutes to complete the DNA extension. Then, the PCR products were analyzed by electrophoresis in a 2% agarose gel and a 472 bp DNA band was observed (FIG. 5).

### Example 6

### Anti-Coagulation Activity of Hirudin in Milk

The anti-coagulation activity of hirudin in mouse milk was measured on the basis of its ability to inhibit the release of colored 4-nitranilin (NA) by thrombin from chromogenic substrate Tos-Gly-Pro-Arg-4-NA (Lyer, L. et al., 1995. Thrombosis Res., Vol. 78 (No.3), pp. 259-263.). Mouse milk was collected and centrifuged at 14,000×g for 5 minutes. The supernatant was mixed with a thrombin solution (50 U/ml) and Tris buffer (50 mM Tris, pH 8.3, 227 mM NaCl). Different volume of supernatant were mixed with the thrombin solution. After incubation at 37° C. for 10 minutes, a chromogenic substrate Tos-Gly-Pro-Arg-4-NA was added and absorbance at 405 nm (A₄₀₅) was read for 1 minute. A commercialized natural hirudin (American Diagnostica Inc. recombinant hirudin #5301, vial of 200 μg ca. 2000 ATU) was used to establish a standard curve. A lower the A₄₀₅ reading value indicates a higher anti-coagulation activity. Based on the standard curve and the reading values, hirudin in milk was quantified.

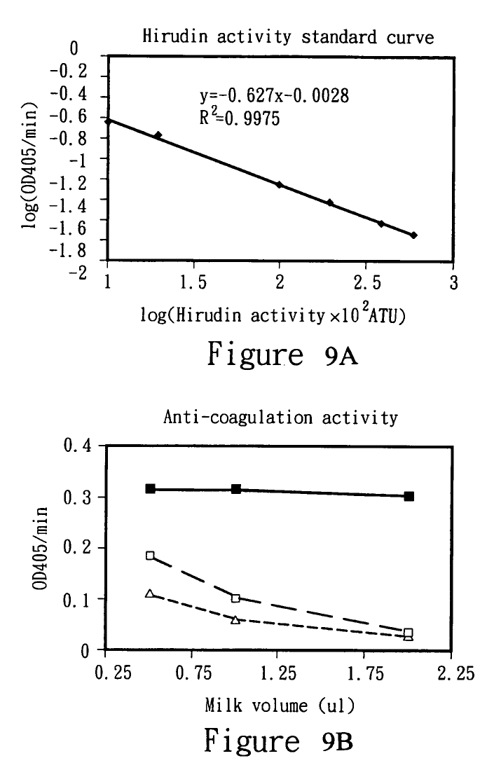

As shown in FIG. 9, milk collected from lactating transgenic mice of the invention has a high level of anti-coagulation activity in comparison with that from non-transgenic mice (normal mouse milk). Milk collected from all 4 transgenic lines expressed an anti-coagulation activity ranging from 0.1 to 40 units per microlitter of milk and sustained during the whole lactation periods (Table 7).

TABLE 7
Anti-coagulation activity of hirudin in milk of transgenic mouse
(ATU/ml)

| Lines | No. of mice | Day 7 | Day 14 | Day 21 |
|---|---|---|---|---|
| 2-1 (F1) | 14 | 12,000-40,000 | 3,000-5,400 | 3,600-4,600 |
| 4-4 (F0) | 1 | 350 | 340 | 480 |
| 6-1 (F1) | 15 | 100-450 | 110-1,100 | 380-1,200 |
| 6-3 (F1) | 15 | 350-3,200 | 2,900-3,600 | 2,200-3,200 |

(F0) Transgenic founder
(F1) First generation of transgenic offspring

As described above, a total of 4 hirudin transgenic mice lines were successfully produced, which have a high level of anti-coagulation activity in their milk extracts. The above results demonstrate that the BC1-GB-Hir construct of the invention can efficiently express a high level of hirudin in mammary gland of the transgenic animals. In addition, the expression period lasting for at least three weeks at high level throughout, especially the 2-1 transgenic line, is beneficial to produce hirudin in a large mount in comparison with the prior art. According to the invention, it is advantageous for producing large amounts of recombinant proteins by using this promoter.

Domestic animals including pig, dairy goat or dairy cattle, which are transgenic with the BC1-GB-Hir construct of the invention, are generated according to the methods described herein to collect hirudin from their milk.

### Example 7

### Hirudin Expression by pE-αLA-Hi in Transformed Mammary Gland Cell Line

7.1 Culture of Mammary Gland Cell Line

The murine mammary gland epidermal cell line NMuMG (CCRC 60087), purchased from the cell bank of the National Health Research Institute (Taipei, Taiwan, ROC), were cultured in Dulbecco Modified Eagles Medium (DMEM) containing 4.5 mg/ml glucose and 10% fetal bovine serum (FBS) at 37° C., 5% CO₂. NMuMG cells grown in the above culture conditions exhibited an appearance of a single polygon without tentacles and did not have the function of differentiated mammary gland cells, e.g., transferring or secreting proteins out of the cell. However, if 5 μg/ml insulin, 5 μg/ml prolactin and 1 μg/ml dexamethasome were added to the culture medium and a layer of Matrixgel (50 ml/cm², purchased from Sigma) was coated on the bottom of the petri dish, after incubation for 24 hours, obvious cell colonies of the NMuMG cells were observed. Each colony included tens of thousands of aggregating cells and formed hollow hemispheres in morphology, which are similar to the appearance of a lactating cell cluster of mammary gland, in vivo.

7.2 Transformation of Mammary Gland Cell Line

The pE-αLA-Hi expression vector (5 μg, 50 μl), described in Example 2, was homogeneously mixed with liposomes (100 μL, SuperFact, QIAGENE) and then serum-free DMEM medium (850 μl) was added to the mixture to generate a “D NA-liposome-medium solution.” NMuMG cells, cultured to a density of 60 to 80% on Matrixgel without adding hormones (insulin, prolactin and dexamethasome), were rinsed with a phosphate-buffered saline (PBS, pH 7.4) for three times. The “DNA-liposome-medium solution” was added to the cells, which were then incubated at 37° C., 5% CO₂ for one hour and 4 ml DMEM medium containing 20% FBS was added thereto. After continuously culturing the cells for 24 hours, the solution containing DNA and liposomes was removed and DMEM medium containing 10% FBS was added. Geneticin (500 μg/ml, G418, Sigma) was also added for selection of transformed cells, and the culture medium was replaced irregularly thereafter. After two successive generations, NMuMG cells stably having the pE-αLA-Hi expression vector in the presence of G418 (pE-αLA-Hi/NMuMG) was obtained.

### Example 8

### Hirudin Expressed by Transformed Mammary Gland Cells Isolated from Mammary Gland Tissue

8.1 Isolation of Mammary Gland Cells from Mammary Gland Tissue

ICR female mice in lactation were sacrificed on the 11^{th} day after delivery, and the mammary gland tissue thereof was isolated. The in vitro mammary gland tissue was rinsed with 1-fold PBS for 3 times and was centrifuged at a low speed of 1,000 rpm for 15 minutes in order to wash off the milk. The mammary gland tissue was cut into 8 mm³ pieces. A piece of 0.25 g tissue was suspended in 0.8 ml DMEM medium.

8.2 Transformation of Mammary Gland Cells

Transformation of the mammary gland cells was carried out by electroporation. The pE-αLA-Hi plasmid DNA (40 μg) was added into the above-described mammary gland tissue. After the mammary gland tissue and DNA were homogenously mixed for 10 minutes, they were put into an electroporation cuvette of 0.4 cm in width and treated by an electroporator (ECM 2001, BTX, USA) under the condition of 200 V/cm, 50 ms for 6 times. The treated mammary gland tissue was moved into a 35 mm petri dish and the medium was changed to DMEM medium containing 5 μg/ml insulin, 5 μg/ml prolactin, 1 μg/ml dexamethasome and 10% FBS.

The transformed mammary gland tissue was cultured in an incubator at 37° C., 5% CO₂ for 48 hours, and then homogenized to obtain a homogenous tissue solution. Determination of anti-coagulation activity of hirudin in the homogeneous extract of the transformed mammary gland tissue was carried out as described above.

8.3 Analysis of Biological Activity of Hirudin in Transformed Mammary Gland Tissue

Determination of anti-coagulation biological activity was carried out as described above with various amounts (0.03, 0.06, 0.43, 0.25, 0.5, 1 and 2 mg total protein) of the homogeneous extract of the transformed mammary gland tissue or various amounts (0.15, 0.31, 0.62, 1.25, 2.5, 5 and 10 μl) of the culture medium.

### Example 9

### Analysis of Biological Activity of Hirudin Expressed by Transformed Mammary Gland Cell Line

9.1 Expression of Hirudin by Mammary Gland Cell Line

The pE-αLA-Hi transformed mammary gland cell line (pE-αLA-Hi/NMuMG) obtained in Example 8 was cultured in the mammary gland cell culture medium containing hormones (insulin, prolactin and dexamethasome) and 500 μg/ml geneticin in a petri dish coated with Matrixgel at 37° C., 5% CO₂. When the cell colonies formed a hollow hemisphere similar to the mammary gland follicle, the cell culture was moved into a clean test tube. The cells were treated with 0.25% trypsin solution and then separated from the petri dish. The cells were collected by centrifugation at 1,000 rpm for 5 minutes. The cells were resuspended in 1 ml hypertension solution (25% sucrose, 1 ml EDTA and Tris-HCl, pH 7.5) and incubated at room temperature for 15 minutes. Then, the cells were broken with a sonicator and centrifuged at 6,000 rpm for 10 minutes to remove cell fragments. The obtained supernatant is a hirudin-containing homogeneous extract of mammary gland cells.

9.2 Determination of Anti-Coagulation Biological Activity

Determination of the anti-coagulation biological activity of hirudin was carried out independently for the above-mentioned homogeneous extract of mammary gland cells and cell culture medium. The total amount of protein in the homogeneous extract of mammary gland cells was determined as the basis and unit of addition of the homogeneous extract to the anti-coagulation biological activity assay.

Bovine thrombin (Sigma) at a concentration of 0.2 pmole in an analysis buffer (0.12 M NaCl, 0.01 M sodium phosphate, 0.01% NaN₃ and 0.1% bovine serum albumin, pH 7.4) was provided. Various concentrations (0.04, 0.08, 0.16, 0.32, 0.64, 1.28 and 2.56 pmole) of a commercially available nature hirudin (Sigma), various amounts (0.03, 0.06, 0.13, 0.25, 0.5, 1 and 2 mg of total protein) of the above-described homogeneous extract of mammary gland cells and various amounts (0.15, 0.31, 0.62, 1.25, 2.5, 5 and 10 μl) of the culture medium were independently mixed with the above-mentioned bovine thrombin (50 μL). After incubation at 24° C. for 1 minute, 100 liter of 10-fold analysis-buffer-diluted human serum was added and mixed to react for 20 seconds. After 15 minutes, the absorbance at A₄₀₅ of the reaction mixture was determined. If the A₄₀₅ reading value is lower, it means that the anti-coagulation level is higher.

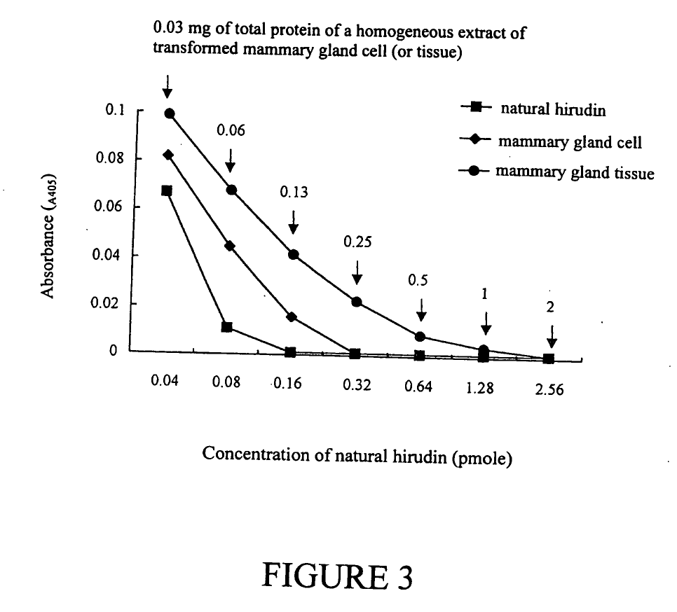

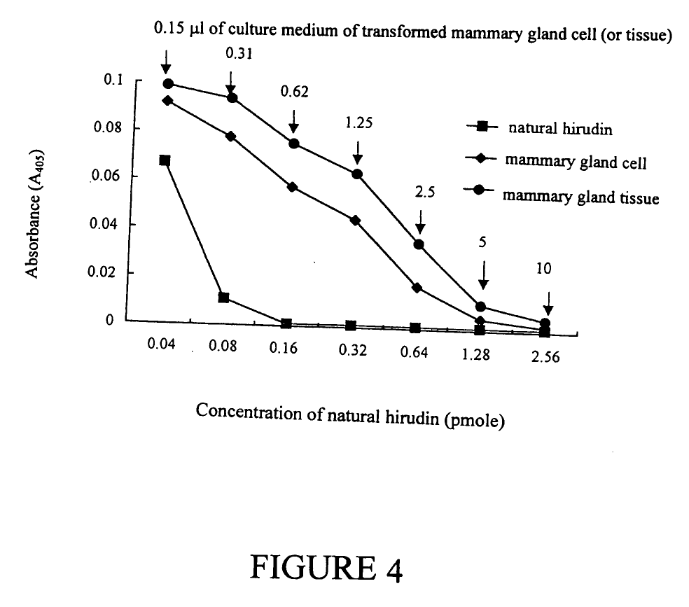

As shown in FIGS. 3 and 4, when the concentration of the nature hirudin is 0.16 pmole, there is almost no coagulation, and a similar no-coagulation situation happened when the amounts of the homogeneous extract of mammary gland cells and the culture medium are 0.25 mg and 10 μl, respectively. The concentrations of the nature hirudin, the homogeneous extract of mammary gland cells and the culture medium needed for the anti-coagulation reaction at the A₄₀₅ reading of 0.05 are 0.52 pmole, 0.056 mg and 0.95 μl, respectively.

1. A nucleic acid construct comprising in operable association a casein gene promoter, a signal sequence and a polynucleotide fragment encoding hirudin.

2. The nucleic acid construct of claim 1, wherein the promoter is isolated from goat β-casein gene.

3. A nucleic acid construct of claim 1, wherein the polynucleotide fragment has a nucleotide sequence of SEQ ID NO: 15 or 16.

4. The nucleic acid construct of claim 1 wherein the signal sequence has a nucleotide sequence of SEQ ID NO: 9.

5. The nucleic acid construct of claim 1, further comprising one or more β-globin insulator elements.

6. A transgenic non-human mammal whose genome comprises the nucleic acid construct of claim 1.

7. The transgenic non-human mammal of claim 6, which is a pig, cattle, horse, goat, camel, sheep, or rodent.

8. The transgenic non-human mammal of claim 6, which is a female and can produce milk that contains hirudin encoded by the polynucleotide fragment encoding hirudin.

9. The transgenic non-human mammal of claim 6, which is male and its female offspring that can produce hirudin encoded by the polynucleotide fragment encoding hirudin.

10. A process for producing hirudin comprising the steps of providing the transgenic non-human mammal of claim 8, collecting milk from the mammal and recovering hirudin from the milk.

11. A process for producing hirudin comprising the steps of providing a male transgenic non-human mammal of claim 9, producing female offspring from the male mammal, collecting milk from the female offspring and recovering hirudin from the milk.

12. An expression vector comprising a replication origin and the nucleic acid construct of claim 1.

13. The expression vector of claim 12, wherein the promoter of the nucleic acid construct is isolated from a β-goat casein gene.

14. The expression vector of claim 12, wherein the polynucleotide fragment of the nucleic acid construct has a nucleotide sequence of SEQ ID NO 16 or 16.

15. The expression vector of claim 12, wherein the signal sequence of the nucleic acid construct has a nucleotide sequence of SEQ ID NO: 9.

16. The expression vector of claim 12, wherein the nucleic acid construct further comprises one or more β-globin insulator elements.

17. (canceled)

18. A transformed mammary gland cell comprising the expression vector of claim 12.

19. The transformed mammary gland cell of claim 18, which is derived from human, pig, cattle, horse, goat, camel, sheep or rodent.

20. A mammalian cell isolated from the transgenic non-human mammal of claim 6, which comprises a genome comprising the nucleic acid construct of claim 1.

21. A process for producing hirudin, comprising the steps of culturing the transformed mammary gland cell of claim 18 under a condition suitable for expressing hirudin and recovering the hirudin therefrom.

22. A process for producing hirudin, comprising the steps of isolating a mammary gland tissue or cell from the transgenic non-human mammal of claim 6, culturing the isolated mammary gland tissue or cell under a condition suitable for expressing hirudin and recovering the hirudin therefrom.
